(19) United States
(12) Patent Application Publication
(10) Pub. No.: US 2018/0237846 A1
(43) Pub. Date: Aug. 23, 2018

(54) GENE SEQUENCING SUBSTRATE, METHOD FOR GENE SEQUENCING, AND GENE SEQUENCING DEVICE

(71) Applicants: BOE TECHNOLOGY GROUP CO., LTD., Beijing (CN); BEIJING BOE OPTOELECTRONICS TECHNOLOGY CO., LTD., Beijing (CN)

(72) Inventors: Yue GENG, Beijing (CN); Peizhi CAI, Beijing (CN); Fengchun PANG, Beijing (CN)

(21) Appl. No.: 15/715,284

(22) Filed: Sep. 26, 2017

(30) Foreign Application Priority Data
Feb. 20, 2017 (CN) 201710090917.9

(51) Int. Cl. C12Q 1/68; B01L 3/00

(52) U.S. Cl. CPC C12Q 1/6869; B01L 2300/0829; B01L 2300/0627; B01L 3/5085

(57) ABSTRACT

Provided is a gene sequencing substrate and its gene sequencing method, and a gene sequencing device. The gene sequencing substrate includes a base substrate; and at least one sequencing unit, disposed on the base substrate, wherein the sequencing unit includes: a reaction cell, configured to accommodate a target sample; and at least one temperature sensor disposed corresponding to the reaction cell and configured to sense a temperature of the reaction cell.

### CROSS REFERENCE

The present application is based upon and claims priority to Chinese Patent Application No. 201710090917.9, filed on Feb. 20, 2017, and the entire contents thereof are incorporated herein by reference.

### TECHNICAL FIELD

The present disclosure relates to a gene sequencing substrate, a method for gene sequencing, and a gene sequencing device.

### BACKGROUND

With the continuous development of gene sequencing technology, gene sequencing technology has gradually become the most commonly used technology in modern molecular biology research, and has a wide application scenarios. Thus, the device used for gene sequencing has a large market space.

Since the development of the first generation gene sequencing in 1977, gene sequencing has achieved considerable development, including the first generation of sanger sequencing technology, the second generation of high-throughput sequencing technology, the third generation of single-molecule sequencing technology, the fourth generation of nano-pore Sequencing technology. The current market mainstream sequencing technology is still the second generation of high-throughput sequencing.

The second-generation high-throughput sequencing technology includes Illumina's sequencing by synthesis technology, Thermo Fisher's ion-semiconductor sequencing technology, sequencing by ligation technology, Roche's pyrosequencing technology and so on, among which the Illumina's sequencing by synthesis technology, by virtue of its ultra-high throughput and relatively long reads, has more than 70% of the market share.

Generally, in the gene sequencing technology, various bases are modified with different fluorescent groups, and when anyone of these bases is paired with a target gene fragment, the fluorescent group will be released. At this time, by detecting the color of the fluorescent light using an optical system, it is possible to determine the type of the base and thus obtain the sequence of the target gene fragment.

It should be noted that, information disclosed in the above background portion is provided only for better understanding of the background of the present disclosure, and thus it may contain information that does not form the prior art known by those ordinary skilled in the art.

### SUMMARY

At least one embodiment of the present disclosure provides a gene sequencing substrate, a method for gene sequencing, and a gene sequencing device.

At least one embodiment of the present disclosure provides a gene sequencing substrate including: a base substrate; and at least one sequencing unit, disposed on the base substrate, wherein the sequencing unit includes: a reaction cell, configured to accommodate a target sample; and at least one temperature sensor disposed corresponding to the reaction cell and configured to sense a temperature of the reaction cell.

At least one embodiment of the present disclosure provides a method for gene sequencing of a gene sequencing substrate including any one of the above gene sequencing substrates, wherein the method for gene sequencing includes: loading a target sample into a reaction cell of the gene sequencing substrate; and adding four different dNTPs successively into the reaction cell and sensing a change of a temperature of the reaction cell.

At least one embodiment of the present disclosure provides a gene sequencing device including: a gene sequencing substrate; an opposite substrate aligned with the gene sequencing substrate to form a flow channel; and a sidewall of the flow channel provided between the gene sequencing substrate and the opposite substrate, wherein the gene sequencing substrate includes any one of the above gene sequencing substrate, and the sidewall of the flow channel surrounds a peripheral of an edge of the gene sequencing substrate to seal the flow channel.

It is to be understood that both the foregoing general description and the following detailed description are exemplary and explanatory only and are not restrictive of the disclosure, as claimed.

This section provides a summary of various implementations or examples of the technology described in the disclosure, and is not a comprehensive disclosure of the full scope or all features of the disclosed technology.

### BRIEF DESCRIPTION OF THE DRAWINGS

In order to more clearly illustrate the technical solutions of the embodiments of the present disclosure, drawings of the embodiments of the present disclosure will be briefly described below. It will be apparent that the drawings in the following description refer only to some embodiments of the present disclosure, and are not intended to limit the present disclosure.

FIG. 1 is a structural schematic diagram of a gene sequencing substrate according to an embodiment of the present disclosure;

FIG. 2 is a structural schematic diagram of another gene sequencing substrate according to an embodiment of the present disclosure;

FIG. 3 is a structural schematic diagram of another gene sequencing substrate according to an embodiment of the present disclosure;

FIG. 4 is a structural schematic diagram of another gene sequencing substrate according to an embodiment of the present disclosure;

FIG. 5 is a structural schematic diagram of another gene sequencing substrate according to an embodiment of the present disclosure;

FIG. 6 is a schematic plan view of a gene sequencing substrate according to an embodiment of the present disclosure;

FIG. 7 is a flow chart of a method for gene sequencing of a gene sequencing substrate according to an embodiment of the present disclosure;

FIG. 8 is a structural schematic diagram of a gene sequencing device according to an embodiment of the present disclosure; and

FIG. 9 is a schematic perspective view of a gene sequencing device according to an embodiment of the present disclosure.

### DETAILED DESCRIPTION

In order to make the objectives, technical solutions and advantages of the embodiments of the present disclosure more clear, the technical solutions of the embodiments of the present disclosure will be described clearly and completely in conjunction with the accompanying drawings of the embodiments of the present disclosure. It is obvious that the described embodiments are part of the embodiments rather than all embodiments of the present disclosure. All other embodiments obtained by one of ordinary skill in the art based on the described embodiments of the present disclosure without the need for creative work are within the scope of the present disclosure.

Unless otherwise defined, technical terms or scientific terms used in the present disclosure should be construed as the general meaning of the understanding of those having ordinary skill in the art to which the present disclosure belongs. The terms “first” and “second” or the like, as used in the present disclosure, do not indicate any order, number or importance, but are merely intended to distinguish between different constituents. The terms “comprise”, “include” or the like indicate that the element or object before the term cover the element or object and their equivalents after the term, without excluding the presence of other elements or objects. The terms “connect”, “interconnect” or the like are not limited to physical or mechanical connections, but may include electrical connections, whether directly or indirectly.

Generally, in the gene sequencing technology, various bases are modified with different fluorescent groups, and the types of the bases are determined by detecting the color of the fluorescent light using an optical system when these bases are paired with the target gene fragment, so that the sequence of the target gene fragment is obtained. However, in this gene sequencing technology, it is necessary to mark the four bases with different fluorescent colors, and the sequencing process requires thousands of rounds of basic pairings, resulting in higher reagent cost for the sequencing, such that the sequencing is costly and thus is not conducive to the promotion and utilization of gene sequencing technology.

During a research, the inventors of the present disclosure have discovered that it is possible to detect the paring of the bases using the fact that the forming of chemical bonds during the base pairing is an exothermic reaction and may cause temperature raise of the reaction system. Accordingly, it is unnecessary to fluorescently mark the four bases, thereby reducing the reagent cost of the gene sequencing and thus the cost of the gene sequencing.

An embodiment of the present disclosure provides a gene sequencing substrate, a method for gene sequencing and a gene sequencing device. The gene sequencing substrate includes a base substrate and at least one sequencing unit provided on the base substrate. The sequencing unit includes a reaction cell configured to accommodate a target sample and at least one temperature sensor disposed corresponding to the reaction cell and configured to sense a temperature of the reaction cell. Accordingly, using the gene sequencing substrate, it is unnecessary to fluorescently mark the four bases with different colors, and by successively loading the four different bases into the reaction cell and detecting the temperature in the reaction cell using the temperature sensor disposed corresponding to the reaction cell, it may determine whether a base pairing has occurred, thereby determining the gene sequencing of the target sample. The gene sequencing substrate has a low reagent cost for the sequencing, such that the cost for sequencing is reduced, and thus is beneficial to the promotion and utilization of gene sequencing technology.

Hereinafter, the gene sequencing substrate, the method for gene sequencing and the gene sequencing device according to embodiments of the present disclosure will be described with reference to the drawings.

### First Embodiment

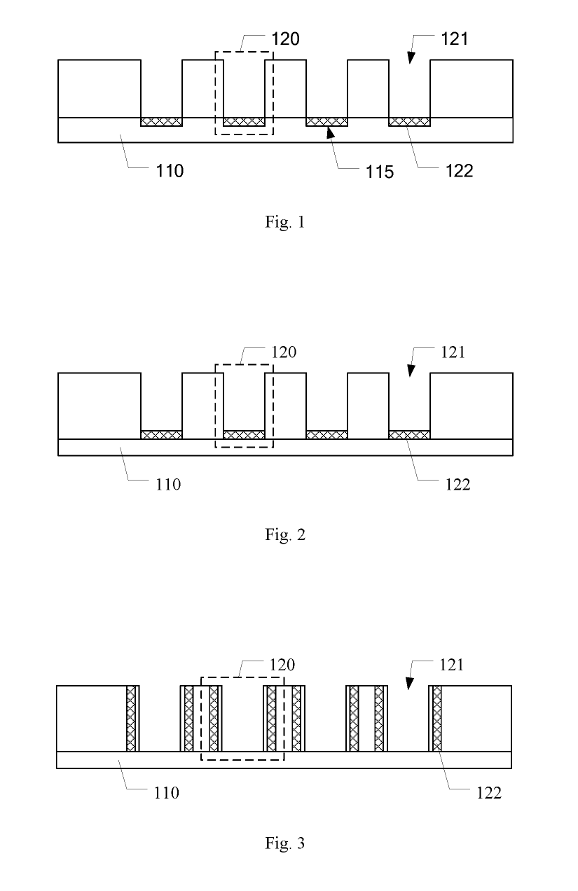

The present embodiment provides a gene sequencing substrate, as illustrated in FIG. 1, the gene sequencing substrate includes a base substrate 110 and at least one sequencing unit 120 disposed on the base substrate 110. In FIG. 1, four sequencing units 120 are illustrated, of course, embodiments of the present disclosure include this but are not limited thereto, and the number of the sequencing unit 120 may be determined according to actual requirement. The respective sequencing unit 120 includes a reaction cell 121 and at least one temperature sensor 122 disposed corresponding to the reaction cell 121. The reaction cell 121 is configured to accommodate a target sample such as a target gene fragment. The at least one temperature sensor 122 disposed corresponding to the reaction cell 121 is configured to sense a temperature of the reaction cell 121. It should be noted that the above corresponding may represent that the temperature sensor may detect the temperature of the reaction cell after being disposed corresponding to the reaction cell. For example, the temperature sensor may be disposed at the bottom or the periphery of the reaction cell, as long as the temperature sensor may measure the temperature of the reaction cell. In addition, the above reaction cell may be formed by a process of forming a film on the base substrate and then undergoing an etch process.

In the gene sequencing substrate according to the present embodiment, when the target sample includes a DNA fragment, four different bases may be loaded into the reaction cell successively, for example, four deoxy-ribonucleoside triphosphates (dNTP) including different bases (e.g., cytosine, guanine, adenine, thymine) may be loaded into the reaction cell successively. The four bases may make contact with the target sample such as the DNA fragment successively, and when the base of the target sample is paired with a currently loaded base, the two bases are bound by phosphodiester bonds to release heat. In this case, it is judged whether or not a base pairing occurs by detecting the temperature of the reaction cell by a temperature sensor disposed corresponding to the reaction cell. If the temperature of the reaction cell rises, it can be judged that the current base (one of the four loaded bases) has a base pairing reaction with the target sample. If the temperature of the reaction cell does not change, it indicates that no base pairing has occurred. After several rounds of the above steps, the gene sequence of the target sample can be determined. Using the gene sequencing substrate, it is possible to perform the gene sequencing without fluorescently marking the four bases with different colors, thereby simplifying the process of the gene sequencing. In addition, the reagent cost for the sequencing using the gene sequencing substrate is relatively low, such that the cost for sequencing is reduced, which is beneficial to the promotion and utilization of gene sequencing technology. Further, the gene sequencing substrate has a simple structure and is easy to operate. It should be noted that, when the target sample includes a RNA fragment, four deoxy-ribonucleoside triphosphates (dNTP) including different bases (e.g., cytosine, guanine, adenine, uracil) may be loaded into the reaction cell successively. The embodiments of the present disclosure include the above, but are not limited thereto.

As illustrated in FIG. 1, for example, in the gene sequencing substrate according to an embodiment of the present disclosure, the at least one sequencing unit may include a plurality of sequencing units. The sequencing units are not overlapped with each other, such that a plurality of target samples may be gene sequenced on the same gene sequencing substrate, thereby improving the sequencing throughput and achieving high-throughput sequencing.

For example, as shown in FIG. 1, each sequencing unit 120 includes a reaction cell 121 and a temperature sensor 122. Of course, the disclosed embodiments include the above, but are not limited thereto, and each sequencing unit may include a plurality of temperature sensors disposed in correspondence with the reaction cell, thereby improving the stability and accuracy of the sequencing unit, avoiding sequencing failure due to error of the temperature sensor.

For example, as shown in FIG. 1, in the gene sequencing substrate provided in an example of the present embodiment, the temperature sensor 122 is disposed at a bottom of the reaction cell 121 adjacent to the base substrate 110. Accordingly, the temperature sensor 122 may correspondingly sense the temperature of the reaction cell 121, and in this case, the area occupied by a sequencing unit 120 on the base substrate 110 is small, so that more sequencing units 120 can be provided on the base substrate 110. Of course, the disclosed embodiments include the above, but are not limited thereto. For example, the temperature sensor may also be provided at other locations, such as the periphery of the reaction cell, i.e. the sidewall of the reaction cell.

For example, as shown in FIG. 1, the temperature sensor 122 may be provided in a recess 115 on the base substrate 110 that is in one to one correspondence to the bottom of the reaction cell 121. Of course, the embodiments of the present disclosure include the above, but are not limited thereto. The temperature sensor may be entirely disposed on the base substrate. As illustrated in FIG. 2, the temperature sensor 122 is provided on an area of the base substrate 110 corresponding to the bottom of the reaction cell 121.

For example, as shown in FIG. 3, in the gene sequencing substrate provided by an example of the present embodiment, the temperature sensor 122 is disposed in the same layer with the reaction cell 121 and at the periphery of the reaction cell 121. Accordingly, the size of the sequencing unit 120 in the direction perpendicular to the base substrate 110 may be reduced, such that the gene sequencing substrate may be slimmer.

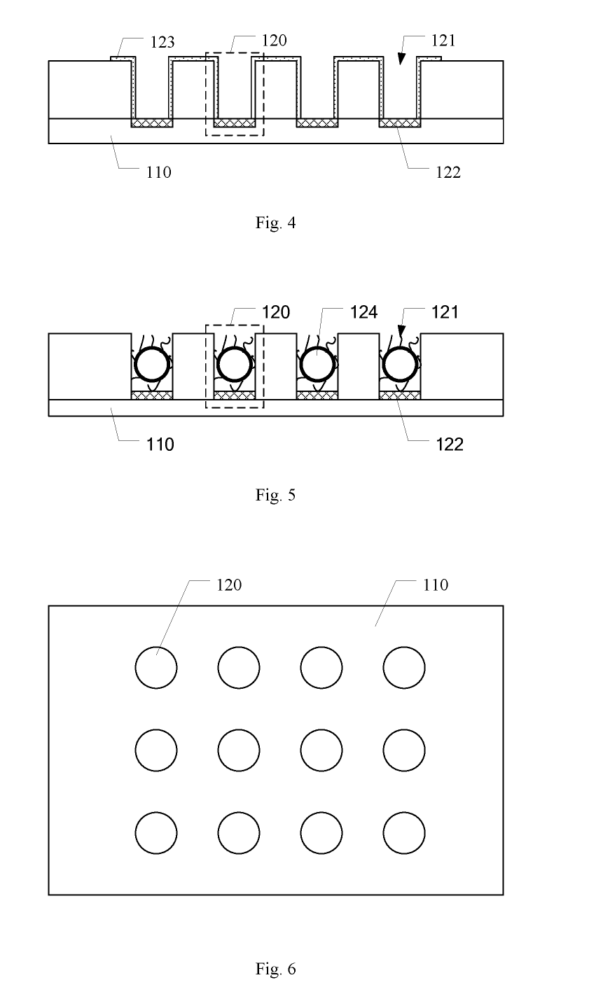

For example, as shown in FIG. 4, in the gene sequencing substrate provided by an example of the present embodiment, the temperature sensor 122 is disposed at the bottom of the reaction cell 121 near the base substrate 110 and the sequencing unit 120 may further include a heat insulation layer 123 disposed on the inner sidewall of the reaction cell 121 to weaken or even eliminate the heat exchange between the adjacent sequencing units 120, thereby further improving the accuracy of gene sequencing results. On the other hand, since the insulating layer is provided on the inner sidewall of the reaction cell, the distance between adjacent reaction cells can be set to be smaller, so that more sequencing units can be provided on the base substrate.

For example, as shown in FIG. 5, in the gene sequencing substrate provided by an example of the present embodiment, the sequencing unit 120 may further include a magnetic bead 124 disposed within the reaction cell 121, and the magnetic beads 124 may adsorb the target sample. Thus, the magnetic beads 124 facilitate the capture and fixation of the target sample.

For example, as shown in FIG. 5, in the gene sequencing substrate provided by an example of the present embodiment, the magnetic beads are provided in one to one correspondence to the reaction cells. By allowing the target sample to be adsorbed on the magnetic beads and loading a magnetic bead in a reaction cell, there is only one kind of target sample in a reaction cell, which improves the accuracy of the gene sequencing results. For example, when the target sample is loaded in the reaction cell by being adsorbed on the magnetic bead, the maximum size of the cross section of the reaction cell can be configured to be larger than the diameter of one magnetic bead and smaller than twice of the diameter of the magnetic bead, such that there is only one magnetic bead in one reaction cell.

It is to be noted that the gene detection substrate provided in the present embodiment may not be provided with magnetic beads, and the target sample is added directly to the reaction cell. For example, a gel layer may be formed at the bottom of the reaction cell and a joint may be provided on the gel layer. The target sample is fixed in the reaction cell by attaching the target sample to the joint of the gel layer in a manner of pairing. The gel layer may be a conventional material, for example, a hydrogel may be included. Further, for example, a material having a gelatinous structure, a material having a polymer mesh structure, or a material having a crosslinked polymer structure may be used. The material having a gelatinous structure may include for example agarose. The material having a polymer mesh structure may include for example gelatin. The material having a crosslinked polymer structure may include for example polyacrylamide. The gel layer may include a material such as silane-free acrylamide or N-[5-(2-bromoacetyl) aminopentyl] acrylamide (BRAPA).

For example, as shown in FIG. 6, in the gene sequencing substrate provided by an example of the present embodiment, a plurality of sequencing units 120 are arranged on the base substrate 110 in an array so as to facilitate the numbering or management of the plurality of sequencing units 120. Of course, the disclosed embodiments include the above, but are not limited thereto, and the plurality of sequencing units may be arranged on the base substrate in other manners.

For example, as shown in FIG. 6, in the gene sequencing substrate provided by an example of the present embodiment, the reaction cell may have a cross sectional shape of circular, regular polygonal, and the like.

For example, in the gene sequencing substrate provided by an example of the present embodiment, a maximum size of the cross section of the reaction cell may be 10-100 μm. By setting the maximum size of the cross section of the reaction cell to be 10-100 it may facilitate loading only one kind of target sample in one reaction cell, such that the gene sequencing result of the gene sequencing substrate may be more accurate. For example, when the target sample is loaded in the reaction cell by adsorption on the magnetic beads, the maximum size of the cross section of the reaction cell can be set to 29 μm, and the diameter of the magnetic beads is set to 20 μm. In this case, a reaction cell can only accommodate one magnetic bead, such that only one kind of target sample is loaded in one reaction cell. It should be noted that when the cross section of the reaction cell is circular, the maximum dimension of the cross section is a circular diameter; and when the cross section of the reaction cell is a regular polygon, the maximum dimension of the cross section is the diagonal of the regular polygon.

For example, in the gene sequencing substrate provided by an example of the present embodiment, the depth of the reaction cell is greater than the maximum dimension of the cross section of the reaction cell, and a ratio between the depth of the reaction cell and the maximum dimension of the cross section of the reaction cell may range from 1.25 to 5. It should be noted that the depth of the reaction cell described above is the distance from the one end of the reaction cell away from the base substrate to the base substrate.

### Second Embodiment

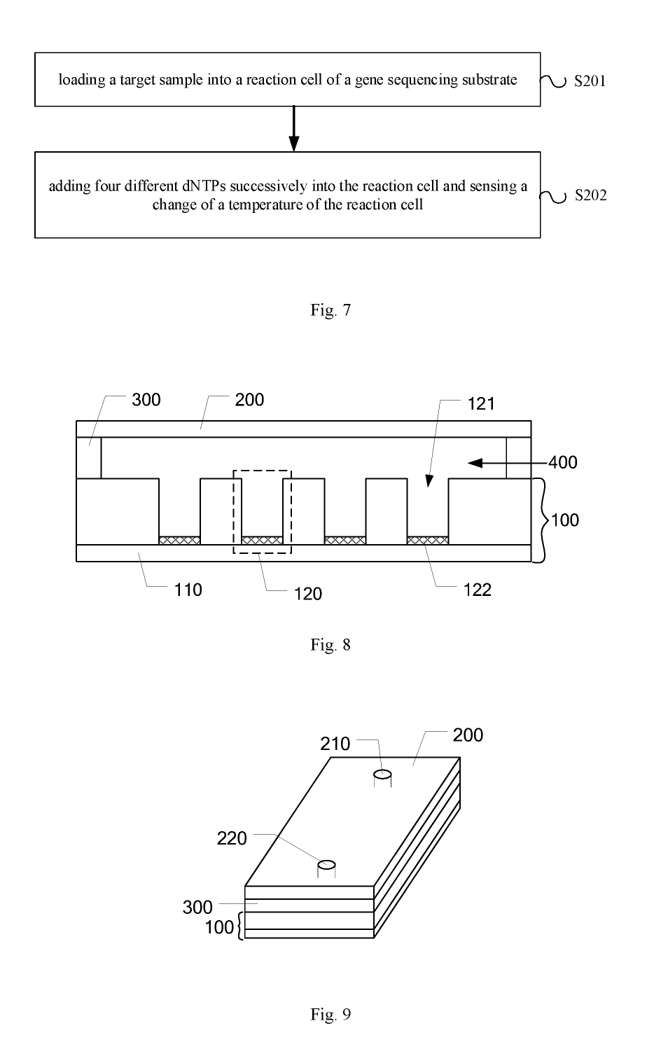

Based on the first embodiment, the present embodiment provides a method for gene sequencing of the gene sequencing substrate. The gene sequencing substrate may be the gene sequencing substrate according to any one of the examples of the above first embodiment. As illustrated in FIG. 7, the method for gene sequencing includes steps S201-S202.

In step S201, a target sample is loaded in the reaction cell.

In step S202, four kinds of dNTPs including different bases are added successively into the reaction cell and a change of the temperature of the reaction cell is sensed using the temperature sensor.

In the method for gene sequencing of the gene sequencing substrate provided by the present embodiment, by adding four kinds of dNTPs including different bases (for example, in the four kinds of dNTPs including different bases, the four bases may include cytosine, guanine, adenine and thymine; or cytosine, guanine, adenine and uracil) into the reaction cell successively, the four different bases may make contact with the target sample such as the DNA fragment in the reaction cell successively. When the base on the target sample is paired with the currently added base, the two bases are bound by phosphodiester bonds to release heat. In this case, it is judged whether or not a base pairing occurs by detecting the temperature of the reaction cell by a temperature sensor disposed corresponding to the reaction cell. If the temperature of the reaction cell rises, it can be judged that the current base (one of the four loaded bases) has a base pairing reaction with the target sample. If the temperature of the reaction cell does not change, it indicates that no base pairing has occurred. After several rounds of the above steps, the gene sequence of the target sample can be determined. Using the method for gene sequencing, it is possible to perform the gene sequencing without fluorescently marking the four basic groups with different colors, thereby simplifying the process of the gene sequencing. In addition, the reagent cost for the sequencing using the method for gene sequencing is relatively low, such that the cost for sequencing is reduced, which is beneficial to the promotion and utilization of gene sequencing technology.

For example, in the method for gene sequencing of the gene sequencing substrate according to an example of the present embodiment, the at least one sequencing unit includes a plurality of sequencing units, and the step of loading the target sample into the reaction cell includes: loading different target samples in the reaction cells of the plurality of sequencing units. Accordingly, it is possible to simultaneously perform gene sequencing on a plurality of different target samples, thereby improving the efficiency of the gene sequencing.

For example, in the method for gene sequencing of the gene sequencing substrate according to an example of the present embodiment, the step of loading the target sample into the reaction cell may further include: performing PCR amplification on the target sample to form a plurality of identical target samples; absorbing the plurality of identical target samples using a magnetic bead; and loading the magnetic bead into the reaction cell. Accordingly, the target sample may be captured and fixed using the magnetic bead. In addition, by attaching the plurality of identical target samples on the magnetic bead and loading the same into the reaction cell, the same base pairing reaction may occur over the plurality of identical samples, so that the thermal effect of the base pairing reaction can be increased, thereby facilitating the sensing of the temperature sensor.

For example, in the method for gene sequencing of the gene sequencing substrate according to an example of the present embodiment, the dNTP may include a reversibly terminating dNTP, and the method for gene sequencing further includes: washing the reversibly terminating dNTP loaded in the reaction cell and adding a sulfhydryl reagent into the reaction cell. After detecting the base type at a position on the target sample (for example, a DNA fragment), it is necessary to wash off the reversibly terminating dNTP loaded in the reaction cell and add the sulfhydryl reagent. It should be noted that, unlike the conventional dNTP, a 3′-terminal of the reversibly terminating dNTP is connected with an azide group which does not form a phosphodiester bond during DNA synthesis and thus will interrupt the DNA synthesis. If the sulfhydryl reagent is added, the azide group breaks and forms a hydroxyl group at the original position. After the addition of the sulfhydryl reagent, the base type detection of the subsequent position can be continued. The detection method is the same as the above method and will not be repeated herein.

For example, when the target sample is a DNA fragment, the above reversibly terminating dNTP may include a reversibly terminating deoxyadenosine triphosphate (dATP), a reversibly deoxythymidine triphosphate (dTTP), a reversibly terminating deoxycytidine triphosphate (dCTP), and a reversibly terminating deoxyguanosine triphosphate (dGTP). If the dNTP added and reacted in the reaction cell is the dATP, the base on the target sample (e.g., the DNA fragment) is the thymine. If the dNTP added and reacted in the reaction cell is the dTTP, the base on the target sample (e.g., the DNA fragment) is the adenine. If the dNTP added and reacted in the reaction cell is the dCTP, the base on the target sample (e.g., the DNA fragment) is the guanine. If the dNTP added and reacted in the reaction cell is the dGTP, the base on the target sample (e.g., the DNA fragment) is the cytosine.

### Third Embodiment

As illustrated in FIG. 8, the present embodiment provides a gene sequencing device including: a gene sequencing substrate 100, an opposite substrate 200, and a sidewall of a flow channel 300. The opposite substrate 200 is aligned with the gene sequencing substrate 100 to form the flow channel 400. The flow channel 400 may be configured to accommodate various reagents for gene sequencing, such as the four different base reagents. The sidewall of the flow channel 300 is provided between the gene sequencing substrate 100 and the opposite substrate 200. The above gene sequencing substrate 100 may include the gene sequencing substrate according to any example of the first embodiment, and the sidewall of the flow channel 300 surrounds a peripheral of an edge of the gene sequencing substrate 100 to seal the flow channel 400.

The gene sequencing device provided by the present embodiment may provide a novel gene sequencing device. In the gene sequencing device, four kinds of bases may be added into the flow channel successively. For example, four dNTPs including different bases (e.g., cytosine, guanine, adenine, thymine) may be added to the flow channel successively, and the four bases flows into the reaction cell through the flow channel successively. The four bases make contact with the target sample in the reaction cell successively, and the occurrence of the base pairing is judged by detecting the temperature of the reaction cell using the temperature sensor provided corresponding to the reaction cell. If the temperature of the reaction cell rises, it can be judged that the current base (one of the four successively loaded bases) has a base pairing reaction with the target sample. After several rounds of the above steps, the gene sequence of the target sample can be determined. Using the gene sequencing device, it is possible to perform the gene sequencing without fluorescently marking the four bases with different colors, thereby simplifying the process of the gene sequencing. In addition, the reagent cost for the sequencing using the gene sequencing device is relatively low, such that the cost for sequencing is reduced, which is beneficial to the promotion and utilization of gene sequencing technology. Further, the gene sequencing device has a simple structure and is easy to operate.

For example, in the gene sequencing device according to an example of the present embodiment, the material of the sidewall of the flow channel may be selected from any of silicon oxide, silicon nitride, and polymeric material. Of course, the disclosed embodiments include, but are not limited thereto, and other materials may be used for the material of the sidewall of the flow channel.

As illustrated in FIG. 9, for example, the gene sequencing device according to an example of the present embodiment further includes a sample inlet 210 and a sample outlet 220 provided on the opposite substrate 220, and the sample inlet 210 and the sample outlet 220 are connected to the flow channel 400. Thus, various reagents for gene sequencing can be added through the sample inlet 210, and the sample outlet 220 is used for the discharge of various waste streams and reagents. It should be noted that the shape of the sample inlet and the sample outlet provided by the present embodiment is not limited to the circular shape shown in FIG. 9, and the shape and size of the sample inlet and the sample outlet can be set according to the actual situation.

The gene sequencing substrate, the method for gene sequencing, and the gene sequencing device according to embodiments of the present disclosure have at least one of the following advantageous effects:

(1) It is possible to perform the gene sequencing without fluorescently marking the four bases with different colors, thereby simplifying the process of the gene sequencing.

(2) The reagent cost for the sequencing is relatively low, such that the cost for sequencing is reduced, which is beneficial to the promotion and utilization of gene sequencing technology.

(3) The gene sequencing substrate and the gene sequencing device have simple structures and are easy to operate.

The following should be noted.

In the drawings of the present disclosure, only the structures related to the embodiments of the present disclosure are involved, and other structures may be referred to the conventional design.

In the event of non-conflict, the features of the same embodiments and different embodiments of the present disclosure may be combined with each other.

Those described above are only the specific embodiments of the present disclosure. However, the scope of the present disclosure is not limited thereto, and any variations or substitutions that is easily conceivable to a person skilled in the art within the technical scope disclosed in this disclosure, are intended to be within the scope of the present disclosure. Accordingly, the scope of protection of the present disclosure should be determined according to the scope of the claims.

What is claimed is:

1. A gene sequencing substrate, comprising:
a base substrate; and
at least one sequencing unit, disposed on the base substrate,
wherein the sequencing unit comprises:
a reaction cell, configured to accommodate a target sample; and
at least one temperature sensor disposed corresponding to the reaction cell and configured to sense a temperature of the reaction cell.

2. The gene sequencing substrate according to claim 1, wherein the temperature sensor is disposed at a bottom of the reaction cell adjacent to the base substrate.

3. The gene sequencing substrate according to claim 1, wherein the temperature sensor is disposed in the same layer with the reaction cell and at a periphery of the reaction cell.

4. The gene sequencing substrate according to claim 2, wherein the sequencing unit further comprises a heat insulation layer disposed on an inner sidewall of the reaction cell.

5. The gene sequencing substrate according to claim 1, wherein the at least one sequencing unit comprises a plurality of sequencing units.

6. The gene sequencing substrate according to claim 5, wherein the plurality of sequencing units are arranged on the base substrate in an array.

7. The gene sequencing substrate according to claim 1, wherein a maximum size of a cross section of the reaction cell is 10 μm-100 μm.

8. The gene sequencing substrate according to claim 7, wherein a depth of the reaction cell is greater than the maximum size of the cross section of the reaction cell.

9. The gene sequencing substrate according to claim 8, wherein a ratio between the depth of the reaction cell and the maximum size of the cross section of the reaction cell ranges from 1.25 to 5.

10. The gene sequencing substrate according to claim 1, wherein the sequencing unit further comprises:
a magnetic bead disposed within the reaction cell, wherein the magnetic bead is configured to adsorb the target sample.

11. The gene sequencing substrate according to claim 10, wherein the magnetic bead is provided in one to one correspondence to the reaction cell.

12. The gene sequencing substrate according to claim 10, wherein the maximum size of the cross section of the reaction cell is configured to be larger than a diameter of one magnetic bead and smaller than twice of the diameter of the magnetic bead.

13. The gene sequencing substrate according to claim 1, wherein the sequencing unit further comprises:
a gel layer, formed at a bottom of the reaction cell and provided with a joint thereon, wherein the target sample is attached to the joint of the gel layer in a manner of pairing.

14. A method for gene sequencing of a gene sequencing substrate, comprising:
loading a target sample into a reaction cell of a gene sequencing substrate; and
adding four different dNTPs successively into the reaction cell and sensing a change of a temperature of the reaction cell.

15. The method for gene sequencing of a gene sequencing substrate according to claim 14 further comprising:
in a case where the temperature of the reaction cell rises, judging that a current base of the added four different dNTPs has a base pairing reaction with the target sample; and
in a case where the temperature of the reaction cell does not change, judging that no base pairing has occurred.

16. The method for gene sequencing of a gene sequencing substrate according to claim 14, wherein the gene sequencing substrate comprises:
a base substrate; and
at least one sequencing unit, disposed on the base substrate,
wherein the sequencing unit comprises:
the reaction cell, configured to accommodate the target sample; and
at least one temperature sensor disposed corresponding to the reaction cell and configured to sense the temperature of the reaction cell.

17. The method for gene sequencing of a gene sequencing substrate according to claim 14, wherein the step of adding four different dNTPs successively into the reaction cell comprises:
performing amplification on the target sample to form a plurality of identical target samples;
absorbing the plurality of identical target samples using a magnetic bead; and
loading the magnetic bead into the reaction cell.

18. The method for gene sequencing of a gene sequencing substrate according to claim 14, wherein the dNTPs comprise reversibly terminating dNTPs, and the method for gene sequencing further comprises:
washing the reversibly terminating dNTP loaded in the reaction cell and adding a sulfhydryl reagent into the reaction cell.

19. A gene sequencing device, comprising:
a gene sequencing substrate;
an opposite substrate aligned with the gene sequencing substrate to form a flow channel; and
a sidewall of the flow channel provided between the gene sequencing substrate and the opposite substrate,
wherein the gene sequencing substrate comprises the gene sequencing substrate according to claim 1, and the sidewall of the flow channel surrounds a peripheral of an edge of the gene sequencing substrate to seal the flow channel.

20. The gene sequencing device according to claim 19, further comprising:
a sample inlet and a sample outlet provided on the opposite substrate and connected to the flow channel.
